(19) United States
(12) Patent Application Publication
(10) Pub. No.: US 2003/0049159 A1
(43) Pub. Date: Mar. 13, 2003

(54) Deodorizer and method of carrying out deodorization

(76) Inventor: Keiichi Onaka, Fukuoka-city (JP)

Correspondence Address: GRIFFIN & SZIPL, PC, SUITE PH-1, 2300 NINTH STREET, SOUTH, ARLINGTON, VA, 22204, US

(21) Appl. No.: 09/949,956

(22) Filed: Sep. 12, 2001

(51) Int. Cl. A61L 9/00; G05B 1/00; G01D 11/26; A62B 7/08

(52) U.S. Cl. 422/5; 422/28; 422/105; 422/119; 422/123; 422/305; 422/306

(57) ABSTRACT

A deodorizer includes (a) a deodorant which decomposes odor, (b) a tank containing the deodorant therein, (c) at least one nozzle which blows out the deodorant downwardly in the form of jet, (d) a conduit connecting the tank to the nozzle, (e) a pump which feeds the deodorant to the nozzle from the tank through the conduit, (f) a chamber which surrounds a distal end of the nozzle and is formed with an air inlet, and (g) a deodorizing tower which is in fluid communication with the chamber and which extends towards the deodorant contained in the tank.

### BACKGROUND OF THE INVENTION

1. Field of the Invention

The invention relates to a deodorizer deodorizing odor-containing air which deodorizer can be installed in a factory and so on and be used in a mobile such as an automobile.

2. Description of the Related Art

A deodorizer using a deodorant deodorizes odor from air under a principle that odor-containing air is caused to make contact with the deodorant.

An efficiency of deodorization is dependent on an area in which odor-containing air makes contact with a deodorant. Accordingly, a conventional deodorizer has made an attempt to increase such an area for enhancing a deodorization efficiency. Typical conventional deodorizers are grouped into a scattering type in which deodorant is formed in powders and scattered to make contact with odor, a filler type in which deodorant is dropped into a filler where the deodorant makes contact with odor, and a bubble type in which odor in the form of bubbles passes through a deodorant.

However, the conventional deodorizers are accompanied with problems of complexity in a structure, a heavy weight, a large size, and an increase in fabrication and running costs.

Specifically, the scattering type deodorizer is unavoidably complex in a structure because the deodorizer has to include means for scattering a deodorant and an absorber which has to have a power source for absorbing odor. The filler type deodorizer has include an absorber having a power source, and is unavoidably heavy because a filler containing a deodorant is quite heavy. The bubble type deodorizer is unavoidably complex in a structure because the deodorizer has to include a bubbler, and a tank for containing a deodorant is unavoidably large because a path in which bubbled order passes through a deodorant has to be long.

### SUMMARY OF THE INVENTION

In view of the above-mentioned problems in the conventional deodorizers, it is an object of the present invention to provide a deodorizer which is small in a size, light, not complex in a structure, and capable of reducing fabrication and running costs, and which can enhance a deodorization efficiency.

In one aspect of the present invention, there is provided a deodorizer including (a) an atomizer for atomizing a deodorant, and (b) a system for contacting odor with the atomized deodorant.

By atomizing a deodorant, the deodorant would have a significantly increased area. This ensures that a rate at which a deodorant makes contact with odor is enhanced.

There is further provided a deodorizer including (a) a deodorant which decomposes odor, (b) a tank containing the deodorant therein, (c) at least one nozzle which blows out the deodorant downwardly in the form of jet, (d) a conduit connecting the tank to the nozzle, (e) a pump which feeds the deodorant to the nozzle from the tank through the conduit, (f) a chamber which surrounds a distal end of the nozzle and is formed with an air inlet, and (g) a deodorizing tower which is in fluid communication with the chamber and which extends towards the deodorant contained in the tank.

The deodorant contained in the tank is fed to the nozzle through the conduit by means of the pump, and blown out in jet through the nozzle towards the deodorizing tower. Gas existing around the nozzle in the chamber is absorbed into the deodorizing tower by virtue of momentum and viscosity of the deodorant jet. Hence, it would be possible to absorb a lot of air containing odor, through the air inlet of the chamber.

This phenomenon is called ejector effect. By virtue of the ejector effect, it would be possible to feed much air into the chamber only by means of the pump for feeding the deodorant, even if the deodorizer is not designed to include an air absorber having a power source used only for the air absorber, and mix the atomized deodorant with the absorbed odor-containing air.

A deodorization efficiency is dependent on both an area at which odor-containing air and a deodorant make contact with each other, and velocities of both the odor-containing air and the deodorant. The deodorant would have a significantly increased area by being atomized, and hence, would have an area at which the deodorant makes contact with odor-containing air. In addition, since the deodorant is blown out in the form of jet, the deodorant together with air has an increased velocity. Hence, while the mixture of the atomized deodorant and odor-containing air is blown through the deodorizing tower, the odor-containing air is effectively deodorized.

The reason why a deodorization efficiency is dependent also on a velocity of deodorant drops is as follows.

Deodorization is phenomenon in which floating odor molecules are absorbed to a solid or fluid deodorant. In other words, deodorization means movement of a material into a deodorant from air. In deodorization, odor concentration in air around the deodorant is reduced, and then, odor existing remote away from the deodorant flows towards the deodorant, and subsequently, is absorbed into the deodorant. The above-mentioned steps are repeatedly carried out in deodorization.

Accordingly, in order to enhance a deodorization efficiency, it would be effective not only to increase an area of a deodorant, but also to facilitate diffusion of odor in atomized deodorant. That is, it would be effective to make turbulent flows in the vicinity of a deodorant, and/or increase a velocity of odor relative to a velocity of a deodorant, in order to enhance a deodorization efficiency.

Froessling's equation with respect to a single ball transfer is as follows.

Sh=2.0+0.55 Sc^{1/3}+Re^{1/2}

In the equation, Sh indicates Sharwood's number, and Re indicates Reynold's number. Sharwood's number Sh may be expressed as follows.

Sh=α/D

In the equation, α indicates a material transfer constant, and D indicates a diffusion coefficient determined in dependence on a material. Hence, if a velocity is increased, both Reynold's number and Sharwood's number are increased, and accordingly, a material transfer constant is also increased.

Since the deodorant is pressurized by the pump and is blown out through the nozzle in the form of jet, a greater amount of a deodorant can be atomized per a unit time than the conventional scattering type deodorizer. Hence, it is possible to increase a surface area of the deodorant, and accordingly, enhance a deodorization efficiency.

Since the deodorizer in accordance with the present invention significantly increases a surface area at which atomized deodorant makes contact with odor-containing air, and automatically absorbs odor-containing air thereinto, the deodorizer makes it no longer necessary to include means for scattering a deodorant and means for absorbing air thereinto separately from odor, both of the means have to include a power source for driving themselves, unlike the conventional scattering type deodorizer. Furthermore, the deodorizer in accordance with the present invention is no longer necessary to include fillers unlike the conventional filler type deodorizer, and to have a bubbler and a large-sized tank for containing a lot of deodorant unlike the conventional bubbler type deodorizer.

Thus, the deodorizer in accordance with the present invention can be fabricated to have a simple structure, to be light, and to be small-sized in reduced fabrication and running costs.

It is preferable that the deodorizing tower is formed at its bottom with an opening such that the opening is located above a level of the deodorant contained in the tank.

When the atomized deodorant is blown out through the deodorizing tower at its bottom, the atomized deodorant makes collision with the deodorant. Hence, most of the atomized deodorant is fluidized, and thus, stored in the tank. Thus, it is possible to avoid the atomized deodorant from being exhausted from the deodorizer together with air, and the deodorant from being reduced with the lapse of time.

For instance, it is preferable that the opening is spaced away from a level of the deodorant by a distance equal to or smaller than 10 cm.

The deodorizer may further include a main body which contains the deodorizing tower, defines the tank therein, and is formed with an air outlet.

The main body surrounds the deodorizing tower, and hence, acts as an exhausting tower. Thus, it is not necessary to separately form an exhausting pipe, ensuring simplification of the deodorizer.

By designing the main body to be long, it would be possible to facilitate the atomized deodorant to be fluidized, avoiding reduction of the deodorant with the lapse of time.

In addition, by arranging the main body and the tank vertically in a line, a space on which the deodorizer is installed can be minimized.

It is preferable that the deodorizer further includes a gas-fluid separator which is arranged in the main body for dividing an inner space of the main body into a first space in which the opening is located, and a second space which is in fluid communication with the air outlet.

For instance, the gas-fluid separator is comprised of a punched plate or a mesh. Hence, the mixture of atomized deodorant and odor-containing air can pass through the gas-fluid separator from the first chamber to the second chamber. When the mixture passes through the gas-fluid separator, the atomized deodorant makes contact with the gas-fluid separator, and thus, is fluidized. The thus fluidized deodorant drops into the tank from the gas-fluid separator. Thus, it is possible to avoid the mixture containing the atomized deodorant, from being exhausted from the deodorizer, ensuring that reduction of the deodorant with the lapse of time can be avoided.

If necessary, the deodorizer may be designed to include a plurality of the gas-fluid separators.

The deodorizer may further include (h) a level sensor which senses a level of the deodorant in the tank, (i) a preliminary tank which preliminarily contains deodorant therein, and (j) a control valve which is turned on or off in response to an output signal transmitted from the level sensor, for supplementing deodorant to the tank from the preliminary tank.

When the sensor detects a fall of a level of the deodorant in the tank, the sensor transmits a signal to the control valve so as to communicate the tank and the preliminary tank with each other. By supplementing a deodorant to the tank from the preliminary tank, it is possible to keep a level of the deodorant constant.

It is preferable that the deodorizer further includes a partition defining an inner tank in the tank such that the inner tank is open just below the deodorizing tower.

The deodorant blown out from the nozzle is first stored in the inner tank. When the inner tank is filled with the deodorant, the deodorant overflows the inner tank, and then drops into the tank surrounding the inner tank. Accordingly, even if the deodorant i8 gradually reduced with the lapse of time, the deodorant is reduced first in the tank, and the inner tank is always filled with the deodorant.

Thus, a distance between the opening of the deodorizing tower and a level of the deodorant in the inner tank can be kept constant without necessity of using the above-mentioned level sensor, preliminary tank and control valve.

It is preferable that the deodorizing tower is tapered such that the deodorizing tower has a greater inner diameter at a lower position, in which case, the deodorizing tower is tapered by an angle equal to or smaller than 10 degrees.

There is further provided a deodorizer including (a) a deodorant which decomposes odor, (b) first to N-th tanks containing the deodorant therein wherein N is an integer equal to or greater than 2, (c) first to N-th nozzles each of which blows out the deodorant downwardly in the form of jet, (d) a conduit connecting the first to N-th tanks to the first to N-th nozzles, (e) a pump which feeds the deodorant to the first to N-th nozzles from the first to N-th tanks through the conduit, (f) first to N-th chambers each of which surrounds a distal end of each of the first to N-th nozzles and is formed with an air inlet, (g) first to N-th deodorizing towers each of which is in fluid communication with the first to N-th chambers, respectively, and each of which extends towards the deodorant contained in the first to N-th tank, respectively, and (h) first to N-th main bodies each of which contains the first to N-th deodorizing towers, respectively, defines the first to N-th tanks therein, and is formed with one air outlet, the air outlet in M-th main body being in fluid communication with the air inlet in (M+1)-th main body wherein M is an integer equal to or greater than 1, but equal to or smaller than (N−1).

In accordance with the above-mentioned deodorizer, odor-containing air passes through the first to N-th deodorizing towers at most, and hence, makes contact with atomized deodorant in a greater area than an area by which the odor-containing air makes contact with atomized deodorant in a single deodorizing tower. Accordingly, it is possible to enhance a deodorization efficiency.

It is preferable that each of the first to N-th deodorizing towers is formed at its bottom with an opening such that the opening is located above a level of the deodorant contained in the first to N-th tanks.

The deodorizer may further include first to N-th gas-fluid separators each of which is arranged in the first to N-th main bodies, respectively, for dividing an inner space of each of the first to N-th main bodies into a first space in which the opening is located, and a second space which is in fluid communication with the air outlet.

The deodorizer may further include (h) a level sensor which senses a level of the deodorant in the first to N-th tanks, (i) a preliminary tank which preliminarily contains deodorant therein, and (j) a control valve which is turned on or off in response to an output signal transmitted from the level sensor, for supplementing deodorant to the first to N-th tanks from the preliminary tank.

The deodorizer may further include first to N-th partitions defining an inner tank in each of the first to N-th tanks such that the inner tank is open just below each of the first to N-th deodorizing towers.

It is preferable that each of the first to N-th deodorizing towers is tapered such that each of the first to N-th deodorizing towers has a greater inner diameter at a lower position, in which case, it is preferable that each of the first to N-th deodorizing towers is tapered by an angle equal to or smaller than 10 degrees.

In another aspect of the present invention, there is provided a method of carrying out deodorization, including the steps of (a) atomizing a deodorant, and (b) contacting odor to the thus atomized deodorant.

There is further provided a method of carrying out deodorization, including the steps of (a) atomizing a deodorant into jet, (b) contacting odor to the thus atomized deodorant and causing the jet to absorb air containing the odor thereinto, and (c) allowing the odor and the atomized deodorant to flow by such a distance that the atomized deodorant can decompose the odor.

The advantages obtained by the aforementioned present invention will be described hereinbelow.

In accordance with the present invention, a deodorant is blown out in jet, and odor-containing air is compulsively absorbed into a chamber by virtue of the ejector effect. Accordingly, it is not necessary for the deodorizer to include an air absorber which has to include a power source to drive the air absorber, unlike the conventional scattering type deodorizer. This ensures simplification in a structure of the deodorizer. Furthermore, it is not necessary for the deodorizer to include an air absorber and a filler unlike the conventional filler type deodorizer, ensuring smaller size and smaller weight than the conventional filler type deodorizer. It is not necessary for the deodorizer to include a bubbler and a large-sized tank unlike the conventional bubbler type deodorizer.

As mentioned above, the deodorizer in accordance with the present invention can be smaller in size and simpler in structure than the conventional deodorizers, and can be fabricated with lower fabrication costs and operated with lower running costs than the conventional deodorizers.

In addition, since a deodorant is atomized and blown out in the form of jet, the atomized deodorant and odor-containing air can be well mixed with each other, and a surface area in which the atomized deodorant and odor-containing air make contact with each other can be significantly increased. This ensures significant enhancement in a deodorization efficiency.

Furthermore, since almost all of the components constituting the deodorizer is vertically arranged, the deodorizer in accordance with the present invention can be installed in a smaller space than the conventional deodorizers.

The above and other objects and advantageous features of the present invention will be made apparent from the following description made with reference to the accompanying drawings, in which like reference characters designate the same or similar parts throughout the drawings.

### BRIEF DESCRIPTION OF THE DRAWINGS

FIG. 1 is a cross-sectional view of the deodorizer in accordance with the first embodiment of the present invention.

FIG. 2 is a perspective view of the gas-fluid separator.

FIG. 3 is a cross-sectional view of the deodorizer in accordance with the second embodiment of the present invention.

FIG. 4 is a cross-sectional view of the deodorizer in accordance with the third embodiment of the present invention.

FIG. 5 is a cross-sectional view of the deodorizer in accordance with the fourth embodiment of the present invention.

### DESCRIPTION OF THE PREFERRED EMBODIMENTS

Preferred embodiments in accordance with the present invention will be explained hereinbelow with reference to drawings.

[First Embodiment]

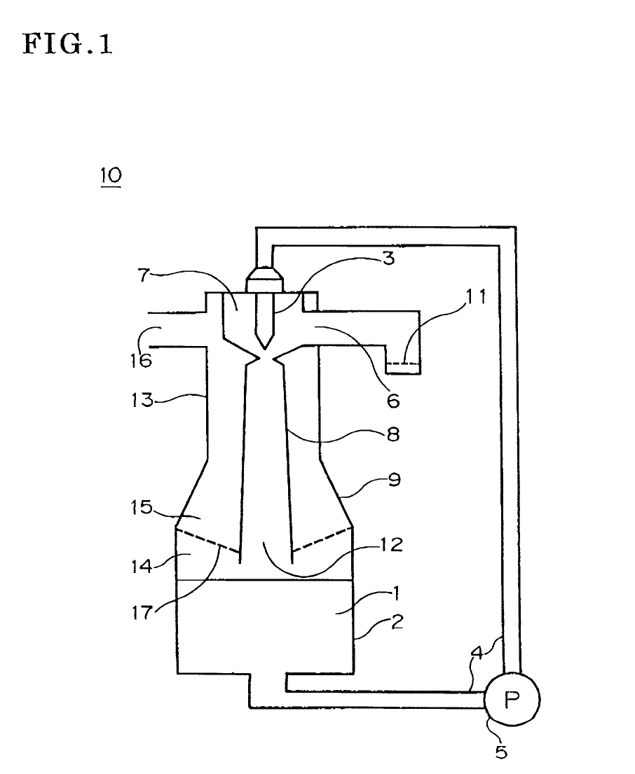

FIG. 1 illustrates the deodorizer 10 in accordance with the first embodiment.

The deodorizer 10 is comprised of a deodorant 1 which decomposes odor, a tank 2 containing the deodorant 1 therein, a nozzle 3 which blows out the deodorant 1 downwardly in the form of jet, a conduit 4 connecting the tank 2 to the nozzle 3, a pump 5 which feeds the deodorant 1 to the nozzle 3 from the tank 2 through the conduit 4, a chamber 7 which surrounds a distal end of the nozzle 3 and is formed with an air inlet 6, a deodorizing tower 8 which is in fluid communication with the chamber 7, extends towards the deodorant 1 contained in the tank 2, and has an opening 12 in facing relation with a level of the deodorant 1, a main body 9 which contains the deodorizing tower 8, defines an exhausting tower 13 around the deodorizing tower 8 and the tank 2 at the bottom, and is formed with an air outlet 16, and a gas-fluid separator 17 which is arranged in the main body 9 for dividing an inner space of the main body 9 into a first space 14 in which the opening 12 is located, and a second space 15 which is in fluid communication with the air outlet 16.

The deodorant 1 contained in the tank 2 is fed to the nozzle 3 from the tank 2 through the conduit 4 by means of the pump 5, and then, is blown out towards the deodorizing tower 8 through a distal end of the nozzle 3.

The nozzle 3 is designed to have a tapered distal end. Hence, a velocity of the deodorant 1 fed to the nozzle 3 is increased in the tapered portion of the nozzle 3, and resultingly, the deodorant 1 is blown out of the nozzle 3 in the form of jet.

The nozzle 3 is surrounded by the chamber 7. The chamber 7 is designed to have a tapered portion at a bottom such that a narrow path defined at the bottom of the tapered portion and connecting the chamber 7 and the deodorizing tower 8 to each other is filled with the deodorant 1 blown out of the nozzle 3.

Accordingly, when the deodorant 1 is blown out of the nozzle 3 in the form of jet, momentum of the jet deodorant 1 is transferred to air, resulting in that air is attracted to the jet deodorant 1. This is so-called ejector effect, as mentioned earlier. Since the chamber 7 is formed with the air inlet 6, a mass of air is attracted into the chamber 7 as the result of the ejector effect.

Thus, the deodorizer 10 in accordance with the first embodiment can attract much odor-containing air through the air inlet 6 without any particular power source for attracting air, and mix the atomized deodorant 1 and odor-containing air to each other.

The air inlet 6 is covered with a filter 11 in order to prevent foreign matters from entering the chamber 7. It should be noted that though the deodorizer 10 is designed to have one air inlet 6, the deodorizer 10 may be designed to have two or more air inlets 6.

The opening 12 of the deodorizing tower 8 is arranged such that it is located slightly above a level of the deodorant 1 contained in the tank 2. The atomized deodorant 1 which deodorizes odor-containing air in the deodorizing tower 8 is fluidized on making contact with a level of the deodorant 1 contained in the tank 2. Thus, the deodorant 1 returns back to the tank 2 without being kept atomized and being exhausted through the air outlet 16 together with air. This ensures less reduction of the deodorant 1 with the lapse of time.

As illustrated in FIG. 1, the deodorizing tower 8 is designed to be tapered such that a lower portion thereof has a greater inner diameter. By designing the deodorizing tower 8 to be tapered, it would be possible to facilitate diffusion of the mixture of the atomized deodorant 1 and odor-containing air.

The deodorizing tower 8 in the first embodiment is tapered by a taper angle of 6 degrees The taper angle is preferably equal to or smaller than 10 degrees.

The gas-fluid separator 17 divides an inner space of the deodorizing tower 9 into the first space 14 and the second space 15. Hence, the atomized deodorant 1 without making contact with a level of the deodorant 1 contained in the tank 2 and directing towards the exhausting tower 13 makes collision with the gas-fluid separator 17, and resultingly, is fluidized. The thus fluidized deodorant 1 falls into the tank 2. Thus, the gas-fluid separator 17 avoids the atomized deodorant 1 from being exhausted through the air outlet 16 together with air having been introduced into the deodorizing tower 8. This ensures less reduction of the deodorant 1 with the lapse of time.

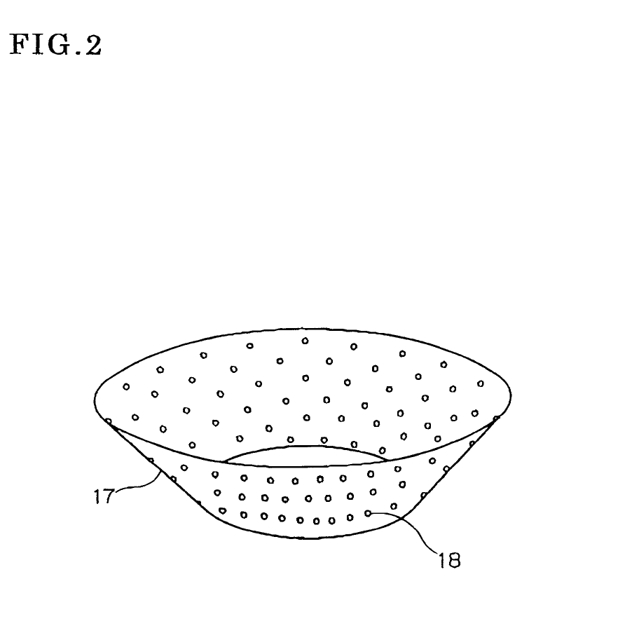

As illustrated in FIG. 2, the gas-fluid separator 17 is punched at its surface to thereby have a lot of small holes 18. Accordingly, the air having been introduced into the deodorizing tower 8 through the air inlet 6 is able to move to the second space 15 from the first space 14 through the gas-fluid separator 17.

As illustrated in FIG. 2, the gas-fluid separator 17 is frusto-conical in shape, that is, has a smaller diameter at a portion closer to its bottom. Accordingly, the deodorant 1 fluidized at a surface of the gas-fluid separator 17 flows downwardly towards the bottom by virtue of gravity, and then, falls into the tank 2.

Though the deodorizer 10 is designed to include one gas-fluid separator 17, the deodorizer 10 may be designed to include two or more gas-fluid separators 17.

The second space 15 is in fluid communication with the air outlet 16. The air having been deodorized in the deodorizing tower 8 and having passed to the second space 15 through the gas-fluid separator 17 is exhausted out of the deodorizer 10 through the air outlet 16.

[Second Embodiment]

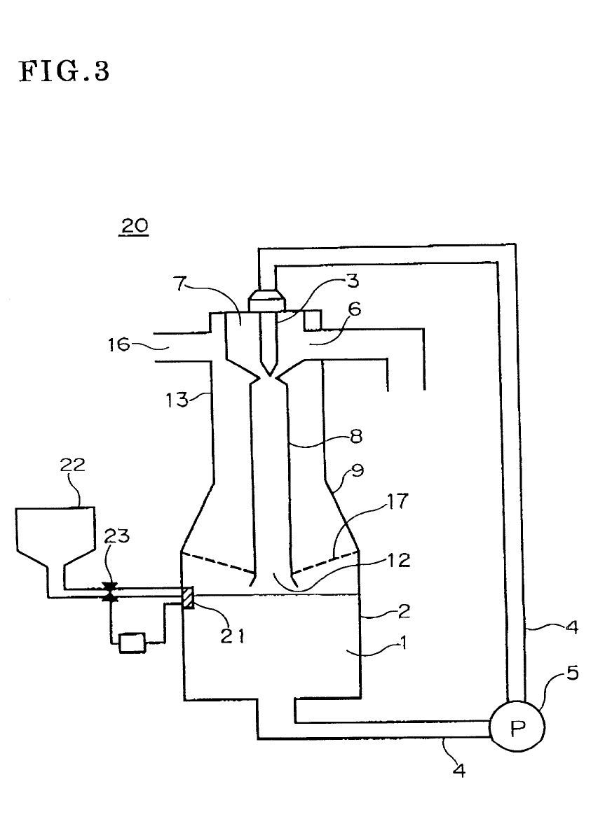

FIG. 3 illustrates the deodorizer 20 in accordance with the second embodiment.

In comparison with the deodorizer 10 in accordance with the first embodiment, illustrated in FIG. 1, the deodorizer 20 further includes a level sensor 21 which senses a level of the deodorant 1 contained in the tank 2, a preliminary tank 22 which preliminarily contains deodorant therein, and a control valve 23 which is turned on or off in response to an output signal transmitted from the level sensor 21, for supplementing deodorant to the tank 2 from the preliminary tank 22. Parts or elements that correspond to those of the first embodiment illustrated in FIG. 1 have been provided with the same reference numerals.

In the deodorizer 20, a level of the deodorant 1 contained in the tank 2 can be kept constant. This ensures reduction in a deodorization efficiency, caused by shortage of the deodorant 1. In addition, a distance between the opening 12 and a level of the deodorant 1 contained in the tank 2 can be kept constant. By optimizing the distance, the atomized deodorant 1 can be effectively fluidized.

[Third Embodiment]

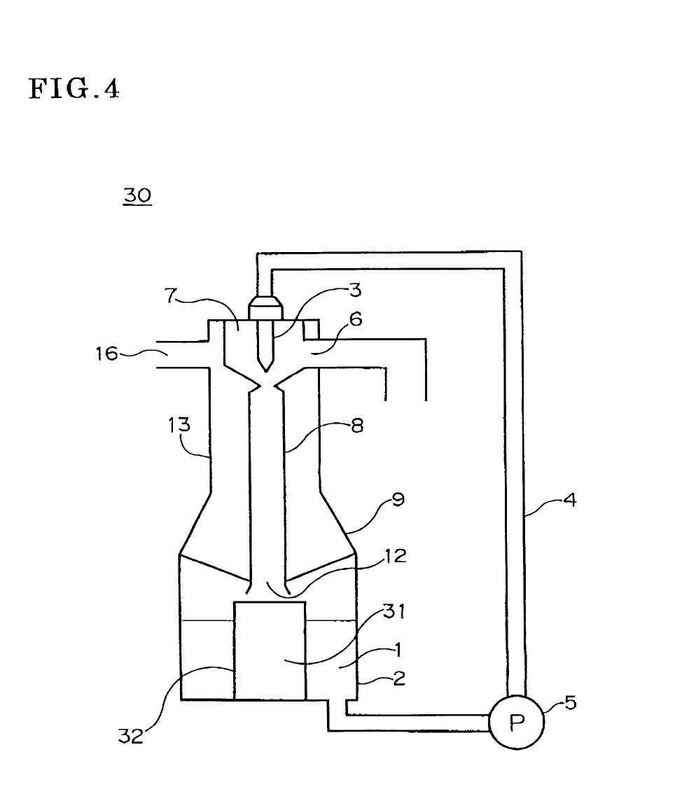

FIG. 4 illustrates the deodorizer 30 in accordance with the third embodiment.

In comparison with the deodorizer 10 in accordance with the first embodiment, illustrated in FIG. 1, the deodorizer 30 further includes a partition 32 defining an inner tank 31 in the tank 2 such that the inner tank 31 is open just below the opening 12 of the deodorizing tower 8.

The deodorant 1 blown out of the deodorizing tower 8 is stored first in the inner talk 31. When the inner tank 31 is filled with the deodorant 1, the deodorant 1 overflows the inner tank 31 and flow into the tank 2. Accordingly, even if the deodorant 1 is gradually reduced with the lapse of time, the deodorant 1 is reduced first in the tank 2, and the inner tank 31 is always filled with the deodorant 1.

Thus, a distance between the opening 12 of the deodorizing tower 8 and a level of the deodorant 1 in the inner tank 31 can be kept constant without necessity for the deodorizer 30 to include the level sensor 21, the preliminary tank 22 and the control valve 23. This ensures that the atomized deodorant 1 blown out of the opening 12 can be effectively fluidized.

[Fourth Embodiment]

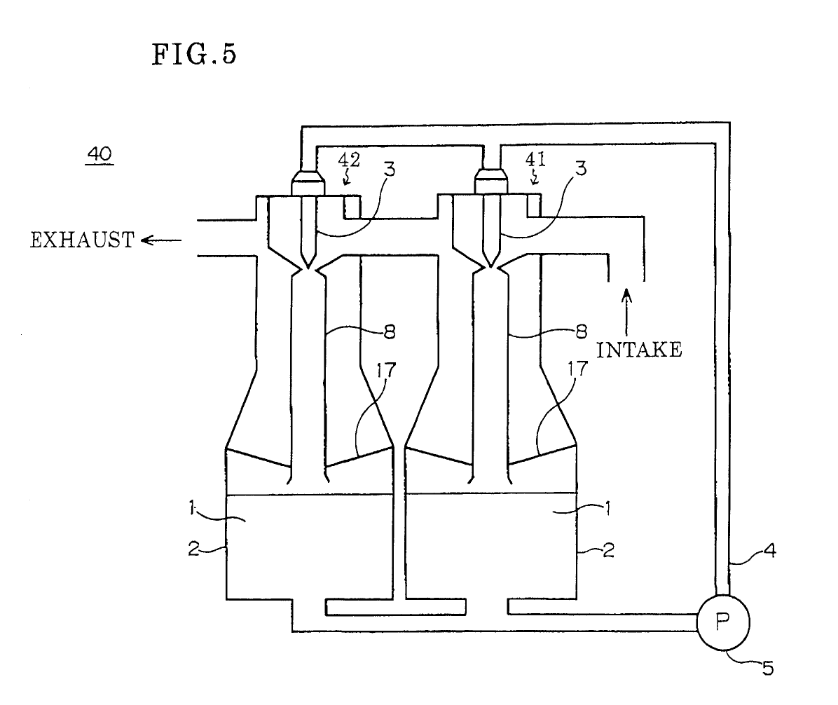

FIG. 5 illustrates the deodorizer 40 in accordance with the fourth embodiment.

The deodorizer 40 is comprised of a first sub-deodorizer 41 and a second sub-deodorizer 42. The first and second sub-deodorizers 41 and 42 are designed to have the same structure as the structure of the deodorizer 10 illustrated in FIG. 1 except the following points.

The conduit 4 connects both of the tanks 2 of the first and second sub-deodorizers 41 and 42 to both of the nozzles 3 of the first and second sub-deodorizers 41 and 42.

The air outlet 16 of the first sub-deodorizer 41 is connected to the air inlet 6 of the second sub-deodorizer 42.

Odor-containing air having been deodorized in the first sub-deodorizer 41 is introduced into the second sub-deodorizer 42 through the air outlet 16 of the first sub-deodorizer 41 and the air inlet 6 of the second sub-deodorizer 42, both of which are connected to each other, and then, deodorized in the second sub-deodorizer 42. Thus, in accordance with the deodorizer 40, odor-containing air is deodorized twice, ensuring enhancement in a deodorization efficiency.

Though the deodorizer 40 is designed to be comprised of the first and second sub-deodorizers 41 ad 42 in the fourth embodiment, the deodorizer 40 may be designed to be comprised of three or more sub-deodorizers, in which odor-containing air is introduced into a first sub-deodorizer, deodorized at N-th times where N indicates the number of sub-deodorizers, and exhausted out of a final sub-deodorizer.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

The entire disclosure of Japanese Patent Application No. 2000-106192 filed on Apr. 7, 2000 including specification, claims, drawings and summary is incorporated herein by reference in its entirety.

What is claimed is:

1. A deodorizer comprising:
(a) means for atomizing a deodorant; and
(b) means for contacting odor with the atomized deodorant.

2. A deodorizer comprising:
(a) a deodorant which decomposes odor;
(b) a tank containing said deodorant therein;
(c) at least one nozzle which blows out said deodorant downwardly in the form of jet;
(d) a conduit connecting said tank to said nozzle;
(e) a pump which feeds said deodorant to said nozzle from said tank through said conduit;
(f) a chamber which surrounds a distal end of said nozzle and is formed with an air inlet; and
(g) a deodorizing tower which is in fluid communication with said chamber and which extends towards said deodorant contained in said tank.

3. The deodorizer as set forth in claim 2, wherein said deodorizing tower is formed at its bottom with an opening such that said opening is located above a level of said deodorant contained in said tank.

4. The deodorizer as set forth in claim 3, further comprising a main body which contains said deodorizing tower, defines said tank therein, and is formed with an air outlet.

5. The deodorizer as set forth in claim 4, further comprising a gas-fluid separator which is arranged in said main body for dividing an inner space of said main body into a first space in which said opening is located, and a second space which is in fluid communication with said air outlet.

6. The deodorizer as set forth in claim 2, further comprising:
(h) a level sensor which senses a level of said deodorant in said tank;
(i) a preliminary tank which preliminarily contains deodorant therein; and
(j) a control valve which is turned on or off in response to an output signal transmitted from said level sensor, for supplementing deodorant to said tank from said preliminary tank.

7. The deodorizer as set forth in claim 2, further comprising a partition defining an inner tank in said tank such that said inner tank is open just below said deodorizing tower.

8. The deodorizer as set forth in claim 2, wherein said deodorizing tower is tapered such that said deodorizing tower has a greater inner diameter at a lower position.

9. The deodorizer as set forth in claim 8, wherein said deodorizing tower is tapered by an angle equal to or smaller than 10 degrees.

10. A deodorizer comprising:
(a) a deodorant which decomposes odor;
(b) first to N-th tanks containing said deodorant therein wherein N is an integer equal to or greater than 2;
(c) first to N-th nozzles each of which blows out said deodorant downwardly in the form of jet;
(d) a conduit connecting said first to N-th tanks to said first to N-th nozzles;
(e) a pump which feeds said deodorant to said first to N-th nozzles from said first to N-th tanks through said conduit;
(f) first to N-th chambers each of which surrounds a distal end of each of said first to N-th nozzles and is formed with an air inlet;
(g) first to N-th deodorizing towers each of which is in fluid communication with said first to N-th chambers, respectively, and each of which extends towards said deodorant contained in said first to N-th tank, respectively; and
(h) first to N-th main bodies each of which contains said first to N-th deodorizing towers, respectively, defines said first to N-th tanks therein, and is formed with one air outlet,
said air outlet in M-th main body being in fluid communication with said air inlet in (M+1)-th main body wherein M is an integer equal to or greater than 1, but equal to or smaller than (N−1).

11. The deodorizer as set forth in claim 10, wherein each of said first to N-th deodorizing towers is formed at its bottom with an opening such that said opening is located above a level of said deodorant contained in said first to N-th tanks.

12. The deodorizer as set forth in claim 10, further comprising first to N-th gas-fluid separators each of which is arranged in said first to N-th main bodies, respectively, for dividing an inner space of each of said first to N-th main bodies into a first space in which said opening is located, and a second space which is in fluid communication with said air outlet.

13. The deodorizer as set forth in claim 10, further comprising:
(h) a level sensor which senses a level of said deodorant in said first to N-th tanks;
(i) a preliminary tank which preliminarily contains deodorant therein; and
(j) a control valve which is turned on or off in response to an output signal transmitted from said level sensor, for supplementing deodorant to said first to N-th tanks from said preliminary tank.

14. The deodorizer as set forth in claim 10, further comprising first to N-th partitions defining an inner tank in each of said first to N-th tanks such that said inner tank is open just below each of said first to N-th deodorizing towers.

15. The deodorizer as set forth in claim 10, wherein each of said first to N-th deodorizing towers is tapered such that each of said first to N-th deodorizing towers has a greater inner diameter at a lower position.

16. The deodorizer as set forth in claim 15, wherein each of said first to N-th deodorizing towers is tapered by an angle equal to or smaller than 10 degrees.

17. A method of carrying out deodorization, comprising the steps of:
(a) atomizing a deodorant; and
(b) contacting odor to the thus atomized deodorant.

18. A method of carrying out deodorization, comprising the steps of:
(a) atomizing a deodorant into jet;
(b) contacting odor to the thus atomized deodorant and causing said jet to absorb air containing said odor thereinto; and
(c) allowing said odor and said atomized deodorant to flow by such a distance that said atomized deodorant can decompose said odor.
